(19) United States
(12) Patent Application Publication
(10) Pub. No.: US 2022/0204655 A1
(43) Pub. Date: Jun. 30, 2022

(54) POLYSACCHARIDES HAVING IMPROVED RADIOCONTRAST PROPERTIES

(71) Applicant: Boston Scientific Scimed, Inc., Maple Grove, MN (US)

(72) Inventors: Joseph T. Delaney, JR., Minneapolis, MN (US); Kolbein Kolste, Acton, MA (US); Tatyana Dyndikova, Minneapolis, MN (US)

(21) Appl. No.: 17/556,408

(22) Filed: Dec. 20, 2021

Related U.S. Application Data
(60) Provisional application No. 63/130,950, filed on Dec. 28, 2020.

(51) Int. Cl. C08B 37/08; A61K 49/04

(52) U.S. Cl. CPC C08B 37/0072; A61K 49/0442

(57) ABSTRACT

The present disclosure pertains to iodinated polysaccharide compounds that comprise a polysaccharide backbone that comprises a plurality of carboxyl groups and a plurality of iodinated side groups. The present disclosure also pertains iodinated polysaccharide compounds in which at least a portion of carboxyl groups that are present in a carboxyl-containing polysaccharide chain are functionalized with a plurality of iodinated side groups. Other aspects of the present disclosure pertain to methods of forming iodinated polysaccharide compounds, medical compositions comprising iodinated polysaccharide compounds, medical procedures comprising introducing such medical compositions into or between tissue of a patient, and medical kits that comprise such medical compositions.

### PRIORITY

The present application is a non-provisional of, and claims the benefit of priority under 35 U.S.C. § 119 to, U.S. Provisional Application Ser. No. 63/130,950, filed Dec. 28, 2020, the disclosure of which is incorporated by reference in its entirety for all purposes.

### FIELD

Among other aspects, the present disclosure relates to medical compositions containing iodinated polysaccharide compounds having radiocontrast properties, to methods of making such iodinated polysaccharide compounds, to medical compositions containing such iodinated polysaccharide compounds, and to medical procedures using such iodinated polysaccharide compounds.

### BACKGROUND

Injectable hydrogels are a newly emerging class of materials having a variety of medical uses. As one specific example, injectable hydrogels have used to create or maintain space between tissues in order to reduce side effects of off-target radiation therapy. The use of a spacer material in conjunction with prostate radiation therapy is illustrated schematically in FIGS. 1A and 1B. FIG. 1A illustrates a cross-section of the human male anatomy including the prostate 110 and rectal wall 112. When the prostate is treated using radiation therapy there is a higher dose region 114 adjacent to the prostate, which is subjected to high doses of radiation, becoming a lower dose region 116 as one proceeds further from the prostate 110. As illustrated in FIG. 1B, a spacing material 118 can be injected between the prostate 110 and the rectal wall 112, which can push the rectal wall from a higher dose region 114 to a lower dose region 116, thereby reducing injury to the rectal wall.

However, for various applications, including the use of injectable hydrogels as spacers near a tumor site prior to radiation therapy, it would be useful for the material to also have some persistent radiocontrast properties. Tungsten particles may be added, but these do not biodegrade, and may settle from the hydrogel, making this a less than ideal strategy. Iodinated contrast can also be added immediately prior to injection. However, this approach poses three problems: 1) an image will need to be taken within hours after implant or the implant will not be visible, 2) follow-up imaging will not have any appreciable contrast, and 3) the iodine is not bound to the hydrogel, so there is a possibility that the contrast that is imaged is not the hydrogel, but rather contrast that has diffused from the hydrogel.

Thus, there is an ongoing need in the biomedical arts for new materials having radiocontrast properties, and for methods of making and using such materials, among other needs.

### SUMMARY

In some aspects, the present disclosure pertains to iodinated polysaccharide compounds that comprise a polysaccharide backbone that comprises a plurality of carboxyl groups and a plurality of iodinated side groups. In certain embodiments, the polysaccharide backbone comprises a carboxyl-containing polysaccharide chain to which the iodinated side groups are attached. In certain embodiments, the carboxyl-containing polysaccharide chain comprises one or more residues selected from one or more of glucuronic acid residues, mannuronic acid residues, or galacturonic acid residues.

In some aspects, the present disclosure pertains to iodinated polysaccharide compounds in which at least a portion of carboxyl groups that are present in a carboxyl-containing polysaccharide chain are functionalized with a plurality of iodinated side groups. In certain embodiments, the carboxyl-containing polysaccharide chain comprises one or more residues selected from glucuronic acid residues, mannuronic acid residues, or galacturonic acid residues.

In some embodiments, which may be used in conjunction with the preceding aspects and embodiments, the iodinated side groups may comprise an iodinated aromatic group in which one or more hydrogens of an aromatic group is substituted by iodine and one or more hydrogens of the aromatic group is substituted by a hydrophilic group. In some of these embodiments, the aromatic group may be a phenyl group. In some of these embodiments, the hydrophilic group may comprise a polyhydroxylated group.

In some embodiments, which may be used in conjunction with the preceding aspects and embodiments, the iodinated side groups may comprise an iodinated aromatic group and a hydrophilic group. In some of these embodiments, the iodinated aromatic side group may comprise a monoiodo-phenyl group, a diiodo-phenyl group, a triiodo-phenyl group or a tetraiodo-phenyl group. In some of these embodiments, the hydrophilic group may comprise a polyhydroxylated group, for example, a polyhydroxylated group that comprises a polyhydroxylated-C₁-C₆-alkyl-containing group, among others.

In some embodiments, which may be used in conjunction with the preceding aspects and embodiments, the iodinated side groups may comprise a 2,4,6-triiodobenzene group in which at least one of the hydrogens at the 3 and 5 positions is substituted by a polyhydroxylated group, for example, a polyhydroxylated group that comprises a polyhydroxylated-C₁-C₆-alkyl-containing group, among others.

In some embodiments, which may be used in conjunction with the preceding aspects and embodiments, the iodinated side groups may comprise an —N,N′-bis(polyhydroxy-C₁-C₆-alkyl)-2,4,6-triiodobenzene-3,5-dicarboxamide group.

In some aspects, the present disclosure pertains to methods of forming an iodinated polysaccharide compound in accordance with any of the preceding aspects and embodiments. In some embodiments, the methods comprise forming an amide linkage by a coupling reaction in which an amino group of an amino-containing iodinated compound is reacted with carboxyl groups of the carboxyl-containing polysaccharide chain.

In some embodiments, which may be used in conjunction with the preceding aspects and embodiments, the coupling is performed in aqueous solution in the presence of a coupling agent. For example, coupling agent may be a carbodiimide coupling agent, among others.

In some embodiments, which may be used in conjunction with the preceding aspects and embodiments, the amino-containing iodinated compound comprises an aromatic group in which one or more hydrogens is substituted by an amino-containing group, one or more hydrogens is substituted by iodine and one or more hydrogens is substituted by a hydrophilic group, for example, a hydrophilic group selected from those described above.

In some aspects, the present disclosure pertains to medical compositions that comprise an iodinated polysaccharide compound in accordance with any of the preceding aspects and embodiments.

In some embodiments, the medical compositions are hydrogels. In certain of these embodiments, the hydrogels are injectable hydrogels.

In some embodiments, which may be used in conjunction with the preceding aspects and embodiments, the medical compositions further comprise a therapeutic agent.

In some aspects, the present disclosure pertains to medical procedures comprising introducing a medical composition in accordance with any of the preceding aspects and embodiments into or between tissue of a patient.

In some embodiments, the medical procedures further comprise imaging the medical composition using an x-ray-based imaging technique.

In some embodiments, which may be used in conjunction with the preceding aspects and embodiments, the medical procedure is selected from a procedure to implant a fiducial marker comprising the iodinated polysaccharide compound, a procedure to implant a tissue regeneration scaffold comprising the iodinated polysaccharide compound, a procedure to implant a tissue support comprising the iodinated polysaccharide compound, a procedure to implant a tissue bulking agent comprising the iodinated polysaccharide compound, a procedure to implant a therapeutic-agent-containing depot comprising the iodinated polysaccharide compound, a tissue augmentation procedure comprising implanting the medical composition, a procedure to introduce the medical composition between a first tissue and a second tissue to space the first tissue from the second tissue.

In some aspects, the present disclosure pertains to medical kits that comprise a medical composition in accordance with any of the preceding aspects and embodiments in a container and one or more of the following: (a) an injectable degradative composition in a container, the degradative composition acting to break down the iodinated polysaccharide compound, (b) a catheter or other delivery device, (d) a needle, or (e) a diluent fluid suitable for injection (e.g., water for injection or saline).

In addition to the above, further aspects and embodiments of the present disclosure will become readily apparent upon review of the Detailed Description to follow.

### BRIEF DESCRIPTION OF THE DRAWINGS

FIGS. 1A and 1B schematically illustrate a cross-section of the human male anatomy including the prostate and rectal wall, before and after injection of a spacer material.

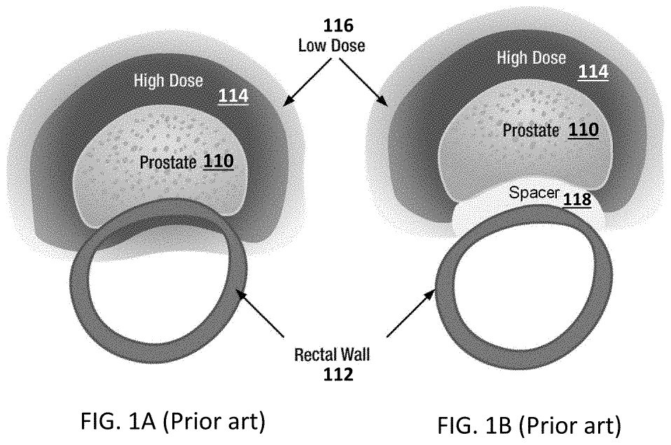

FIG. 2 schematically illustrates a method of forming a radiopaque polysaccharide compound, in accordance with an embodiment of the present disclosure.

### DETAILED DESCRIPTION

In various aspects, the present disclosure provides iodinated polysaccharide compounds that comprise a polysaccharide backbone that comprises a plurality of carboxyl groups and a plurality of iodinated side groups. In various embodiments, the polysaccharide backbone comprises a carboxyl-containing polysaccharide chain to which the iodinated side groups are attached. In various aspects, the present disclosure provides iodinated polysaccharide compounds that in which at least a portion of the carboxyl groups that are present in a carboxyl-containing polysaccharide chain are functionalized with a plurality of iodinated side groups.

By attaching the iodinated side groups to the polysaccharide chain, radiopacity is imparted to the polysaccharide chain, making the iodinated polysaccharide compounds useful, for example, in radiocontrast settings.

The carboxyl-containing polysaccharide chains can generally be any carboxyl-containing polysaccharide of natural origin, synthetic origin or a combination thereof. Particular examples of carboxyl-containing polysaccharide chains include the following: polymers that contain glucuronic acid residues including polyglucuronic acid homopolymers and polyglucuronic acid copolymers such as hyaluronic acid (which comprises D-glucuronic acid residues and N-acetyl-D-glucosamine residues) and various carboxyl-containing gums including gums having glucuronic acid residues such as gellan gum (which comprises D-glucuronic acid residues, D-glucose residues and L-rhamnose residues) and xanthan gum (which comprises D-glucuronic acid residues, D-glucose residues and D-mannose residues); polymers that contain mannuronic acid residues including polymannuronic acid homopolymers and polymannuronic acid copolymers such as alginic acid (which comprises D-mannuronic acid residues and L-glucuronic acid residues); and polymers that contain galacturonic acid residues including polygalacturonic acid homopolymers and polygalacturonic acid copolymers including members of the pectin family which, in addition to D-galacturonic acid residues, can also comprise D-glucuronic acid residues and one or more additional residues selected from D-xylose residues (e.g., xylogalacturonan), D-apiose residues (e.g., apiogalacturonan), α-L-rhamnose residues (rhamnogalacturonan pectins), D-galacturonic acid residues, D-galactose residues, L-arabinose residues, and D-xylose residues). Other particular examples of carboxyl-containing polysaccharide chains include carboxylated cellulose, carboxymethylcellulose, carboxylated starch, carboxymethyl starch, N-carboxymethylchitosan, or N,O-carboxymethylchitosan.

In various embodiments, the iodinated side groups of the iodinated polysaccharide compounds comprise an iodinated aromatic group in which one or more hydrogens of an aromatic group is substituted by iodine and one or more hydrogens of the aromatic group is substituted by a hydrophilic group. In these embodiments, the aromatic group may be selected from a phenyl group or a naphthalene among others.

In various embodiments, the iodinated side groups comprise an iodinated aromatic group and at least one hydrophilic group. In these embodiments, the iodinated aromatic group may be selected from an iodinated phenyl group or an iodinated naphthalene group, among others. In particular embodiments, the iodinated aromatic groups may comprise a monoiodo-phenyl group, a diiodo-phenyl group, a triiodo-phenyl group or a tetraiodo-phenyl group. In certain embodiments, the iodinated side groups may comprise a 2,4,6-triiodobenzene group in which at least one of the hydrogens at the 3 and 5 positions is substituted by a hydrophilic group.

The at least one hydrophilic group may be selected, for example, from polyhydroxylated groups, among others. For example, the at least one polyhydroxylated group may comprise a polyhydroxylated-C₁-C₆-alkyl-containing group, for example, or a polyhydroxylated-C₁-C₆-alkyl-carboxamido group.

In certain embodiments, the iodinated side groups may comprise —N,N′-bis(polyhydroxy-C₁-C₆-alkyl)-2,4,6-triiodobenzene-3,5-dicarboxamide groups, of which —N,N′-bis(2,3-dihydroxypropyl)-2,4,6-triiodobenzene-3,5-dicarboxamide is an example.

Other aspects of the present disclosure are directed to methods of forming iodinated polysaccharide compounds such as those described above. In some embodiments, these methods comprise the formation of an amide linkage, which links the iodinated side groups to the polysaccharide backbone, by a coupling reaction in which an amino group of an amino-containing iodinated compound is reacted with carboxyl groups of a carboxyl-containing polysaccharide chain. By coupling the amino-containing iodinated compound with the polysaccharide, radiopacity is imparted to the polysaccharide.

This coupling can be facilitated in aqueous and non-aqueous solutions with the use of coupling agents. Suitable coupling agents may be selected, for example, from the following: (a) carbodiimides such as 1-ethyl-3-(3-dimethyl-aminopropyl) carbodiimide hydrochloride (EDC), dicyclohexylcarbodiimide (DCC), diisopropylcarbodiimide (DIC), 1-cyclohexyl-3-(2-morpholinyl-4-ethyl) carbodiimide methyl p-toluene sulfonate (CMC), or 1-cyclohexyl-3-(2-morpholioethyl)carbodiimide metho-4-toluenesulfonate (CDI), (b) phosphonium reagents such as BOP (benzotriazol-1-yloxy-tris(dimethylamino)-phosphonium hexafluorophosphate), PyBOPR (benzotriazol-1-yloxy-tripyrrolidino-phosphonium hexafluorophosphate), PyBrOPR (bromo-tripyrrolidino-phosphonium hexafluorophosphate), PyAOP (7-aza-benzotriazol-1-yloxy-tripyrrolidinophosphonium hexafluorophosphate), PyOxim (ethyl cyano(hydroxyimino)acetato-O₂)-tri-(1-pyrrolidinyl)-phosphonium hexafluorophosphate). DEPBT (3-(diethoxy-phosphoryloxy)-1,2,3-benzo[d] triazin-4(3H)-one), (c) aminium/uronium-imonium reagents such as 2-(1H-benzotriazol-1-yl)-N,N,N′,N′-tetramethylaminium tetrafluoroborate/hexafluorophosphate (TBTU, BF₄⁻ anion)/HBTU, PF₆⁻ anion), HCTU (2-(6-chloro-1H-benzotriazol-1-yl)-N,N,N′,N′-tetramethylaminium hexafluorophosphate), HDMC (N-[(5-chloro-1H-benzotriazol-1-yl)-dimethylamino-morpholino]-uronium hexafluorophosphate N-oxide), 2-(7-aza-1H-benzotriazol-1-yl)-N,N,N′,N′-tetramethylaminium tetrafluoroborate/hexafluorophosphate (TATU, BF₄⁻ anion/HATU, PF₆⁻ anion), COMU (1-[1-(cyano-2-ethoxy-2-oxoethylideneaminooxy)-dimethylamino-morpholino]-uronium hexafluorophosphate), TOTT (2-(1-oxy-pyridin-2-yl)-1,1,3,3-tetramethylisothiouronium tetrafluoroborate), TFFH (tetramethylfluoroformamidinium hexafluorophosphate), (d) additional coupling agents such as EEDQ (N-ethoxycarbonyl-2-ethoxy-1,2-dihydroquinoline), T3P (2-propanephosphonic acid anhydride), DMTMM and related compounds (4-(4,6-dimethoxy-1,3,5-triazin-2-yl)-4-methylmorpholinium salts), BTC (bis-trichloromethylcarbonate or “triphosgene”), and CDI (1,1′-carbonyldiimidazole). Additives are commonly used in amide bond formations with carbodiimides, in order to enhance the reactivity and also to reduce formation of epimers as well as N-acylureas. Additives include HOBt (1-hydroxybenzotriazole), HOBt-6-sulfonamidomethyl resin.HCl (1-Hydroxybenzotriazole-6-sulfonamidomethyl resin.HCl), HOOBt (HODhbt) (hydroxy-3,4-dihydro-4-oxo-1,2,3-benzotriazine), HOSu (N-hydroxysuccinimide), HOAt (1-hydroxy-7-aza-1H-benzotriazole), Oxyma Pure (ethyl 2-cyano-2-(hydroximino)acetate), DMAP (4-(N,N-dimethylamino)pyridine). The preceding coupling agents and additives are available, for example, from suppliers such as Bachem Americas, Inc., Torrance, Calif., USA.

Carboxyl-containing polysaccharide compounds for use in such coupling methods may be selected from the polyglucuronic acid homopolymers and copolymers, polymannuronic acid homopolymers and copolymers, polygalacturonic acid homopolymers and copolymers, carboxylated cellulose, carboxymethylcellulose, carboxylated starch, carboxymethyl starch, N-carboxymethylchitosan, or N,O-carboxymethylchitosan, as described above.

In various embodiments, the amino-containing iodinated compound may be water soluble.

In various embodiments, the amino-containing iodinated compound may be a water-soluble iodinated aromatic amine, for example, an iodinated aromatic amine substituted with one or more hydrophilic groups.

In various embodiments, the amino-containing iodinated compound may comprise an aromatic group in which one or more hydrogens is substituted by an amino-containing group, one or more hydrogens is substituted by iodine and one or more hydrogens is substituted by a hydrophilic group. For example, the amino-containing iodinated compound may comprise a benzene group in which at least one of the hydrogens is substituted by an amino-containing group, at least one of the hydrogens is substituted by an iodine group, and least one of the hydrogens is substituted by a hydrophilic group.

In various embodiments, the amino-containing iodinated compound may comprise an iodinated aromatic group in which one or more hydrogens is substituted by an amino-containing group and one or more hydrogens is substituted by a hydrophilic group. For example, the amino-containing iodinated compound may comprise an iodinated benzene group in which at least one of the hydrogens is substituted by an amino-containing group and least one of the hydrogens is substituted by a hydrophilic group. In certain embodiments, the amino-containing iodinated compound may comprise a 2,4,6-triiodobenzene group in which at least one of the hydrogens at the 1, 3 and 5 positions is substituted by an amino-containing group and least one of the hydrogens at the 1, 3 and 5 positions is substituted by a hydrophilic group.

Examples of hydrophilic groups include polyhydroxylated groups, among others.

Particular amino-containing iodinated compounds include 5-amino-N,N′-bis(polyhydroxy-C₁-C₆-alkyl)-2,4,6-triiodobenzene-1,3-dicarboxamide compounds, of which 5-amino-N,N′-bis(2,3-dihydroxypropyl)-2,4,6-triiodobenzene-1,3-dicarboxamide is an example.

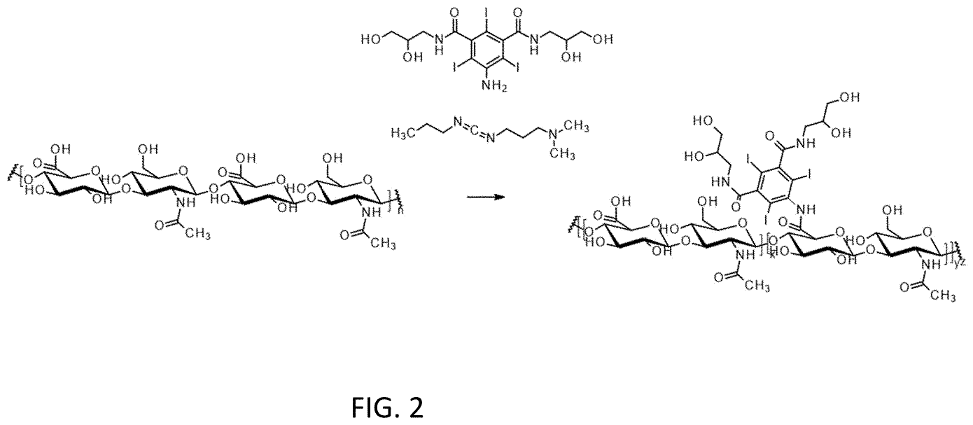

A particular example of a method of forming an iodinated polysaccharide in accordance with the present disclosure will now be described in which at least a portion of the carboxyl groups that are present in a hyaluronic-acid-containing polysaccharide chain are functionalized with iodinated side groups in order to impart radiopacity to the polysaccharide chain. In one beneficial embodiment, carboxyl groups of non-animal stabilized hyaluronic acid (NASHA) are functionalized with iodinated side groups. In sufficiently high concentrations, NASHA solutions form physically crosslinked hydrogels, which are suitable for injection with good biocompatibility. Additionally, NASHA hydrogels have additional attractive features in that they can be readily dissolved under mild conditions by administering hyaluronidase to catalyze their hydrolysis. The NASHA polymer is functionalized with water soluble iodinated side groups. With reference to FIG. 2, the available amino group of the compound 5-Amino-N,N′-bis(2,3-dihydroxypropyl)-2,4,6-triiodoisophthalamide (CAS #76801-93-9) can be coupled to carboxyl groups of the D-glucuronic acid subunits of the NASHA, affording a water soluble, radiopaque moiety. This coupling can be facilitated in aqueous solution with a suitable coupling agent such as EDC.

Other aspects of the present disclosure pertain to compositions that comprise the iodinated polysaccharide compounds of the present disclosure. Compositions that comprise the iodinated polysaccharide compounds of the present disclosure may be used in a wide variety of biomedical applications, including use in injectables, implants and medical devices.

Such compositions include hydrogel compositions that comprise the iodinated polysaccharide compounds of the present disclosure and water. Hydrogels in accordance with the present disclosure may be physically or chemically (e.g., covalently) crosslinked. In some embodiments, hydrogels in accordance with the present disclosure may form lubricious coatings. In some embodiments, hydrogels in accordance with the present disclosure may be injectable hydrogels.

As previously noted, by attaching iodinated side groups to carboxyl-containing polysaccharide chains, radiopacity is imparted to the polysaccharide chains, resulting in iodinated polysaccharide compounds that are useful in radiocontrast settings.

In some embodiments, compositions that comprise the iodinated polysaccharide compounds of the present disclosure may comprise one or more therapeutic agents, such as small molecule drugs, cells, proteins, and bioactive molecules.

In some embodiments, the therapeutic agent may be selected from the following: anesthetics; analgesics, selected from acetaminophen, ibuprofen, flurbiprofen, ketoprofen, Voltaren®, phenacetin and salicylamide; anti-inflammatories selected from naproxen and indomethacin; antihistamines, selected from chlorpheniramine maleate, phenindamine tartrate, pyrilamine maleate, doxylamine succinate, phenyltoloxamine citrate, diphenhydramine hydrochloride, promethazine, brompheniramine maleate, dexbrompheniramine maleate, clemastine fumarate and triprolidine; antitussives selected from dextromethorphan hydrobromide and guaifenesin; expectorants; decongestants, selected from phenylephrine hydrochloride, phenylpropanolamine hydrochloride, pseudoephedrine hydrochloride, and ephedrine; antibiotics selected from amebicides, broad and medium spectrum, fungal medications, monobactams and viral agents; bronchodilators selected from theophylline, albuterol and terbutaline; cardiovascular preparations selected from diltiazem, propranolol, nifedipine, clonidine, alpha adrenoceptor agonists, alpha receptor blocking agents, alpha and beta receptor blocking agents, antiotensin converting enzyme inhibitors, beta blocking agents, calcium channel blockers, and cardiac glycosides; central nervous system drugs selected from thioridazine, diazepam, meclizine, ergoloid mesylates, chlorpromazine, carbidopa and levodopa; metal salts selected from potassium chloride and lithium carbonate; minerals selected from the group consisting of iron, chromium, molybdenum and potassium; immunomodulators; immunosuppressives selected from minocycline, cyclosporine A; thyroid preparations selected from synthetic thyroid hormone, and thyroxine sodium; peptide and glycoprotein hormones and analogues selected from human chorionic gonadotrophin (HCG), corticotrophin, human growth hormone (HGH-Somatotropin) erythropoietin (EPO), basic fibroblast growth factor (FGF), including FGF1 and FGF2, vascular endothelial growth factor (VEGF), platelet derived growth factor (PDGF), angiopoietin 1, and angiopoietin 2; steroids and hormones selected from ACTH, anabolics, androgen and estrogen combinations, androgens, corticoids and analgesics, estrogens, glucocorticoid, gonadotropin, gonadotropin releasing, hypocalcemic, menotropins, parathyroid, progesterone, progestogen, progestogen and estrogen combinations, somatostatin-like compounds, urofollitropin, vasopressin, methyl prednisolone, GM1 ganglioside, cAMP, and others; vitamins selected from water-soluble vitamins and veterinary formulations; growth factors selected from EGF, FGF2 and neurotrophin; peptides, peptide mimetics and other protein preparations; DNA; and, small interfering RNAs.

Examples of settings in which injectable hydrogels in accordance with the present disclosure may be used include injection to provide spacing between tissues, injection (e.g., in the form of blebs) to provide fiducial markers, injection for tissue augmentation or regeneration, injection as a filler or replacement for soft tissue, injection to provide mechanical support for compromised tissue, injection as a scaffold, injection as a carrier of therapeutic agents in the treatment of diseases and cancers and the repair and regeneration of tissue, among others.

The present invention encompasses various ways of administering the compositions of the present disclosure in conjunction with a variety of medical procedures. One skilled in the art can determine the most desirable way of administering the compositions, depending on the type of treatment and the condition of the patient, among other factors. Methods of administration include, for example, percutaneous techniques as well as other effective routes of administration. For example, the compositions of the invention may be delivered through a syringe or through a catheter, for instance, a microcatheter, which can be advanced over a guidewire, a steerable microcatheter, or a flow-directed microcatheter, among other devices.

In various aspects, medical procedures are provided in which a medical composition that comprises an iodinated polysaccharide compound of the present disclosure is inserted into or between tissue of a patient. In various embodiments, the injected medical composition is then imaged using an external or internal imaging technique. Typically, the imaging techniques is an x-ray-based imaging technique, such as computerized tomography or X-ray fluoroscopy.

In certain embodiments, the medical procedure may be one of the following: a procedure to implant a fiducial marker comprising the iodinated polysaccharide, a procedure to implant a tissue regeneration scaffold comprising the iodinated polysaccharide, a procedure to implant a tissue support comprising the iodinated polysaccharide, a procedure to implant a tissue bulking agent comprising the iodinated polysaccharide, a procedure to implant a therapeutic-agent-containing depot comprising the iodinated polysaccharide, a tissue augmentation procedure comprising implanting the medical composition, a procedure to introduce the medical composition between a first tissue and a second tissue to space the first tissue from the second tissue.

Compositions (e.g., hydrogels) in accordance with the present disclosure may be injected at various sites in various medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In other aspects, the present disclosure pertains to medical kits that include a composition that comprises an iodinated polysaccharide compound in accordance with the present disclosure in a suitable container. The composition comprising the iodinated polysaccharide may be in dried form (e.g., in the form of dried particles) or in the form a pre-made hydrogel. The container for the composition comprising the iodinated polysaccharide may be, for example, a vial or a syringe barrel. The syringe barrel may have an opening to receive a plunger at its proximal end and have a fitting (e.g., a luer fitting or another suitable fitting) at its distal tip for direct or indirect engagement with an injection needle or a catheter such that the interior of the syringe barrel is placed in fluid communication with the interior of the injection needle the catheter. The barrel may also be provided with a flange at its proximal end for ease of engagement and a scale for determining the volume of fluid remaining in the barrel. Suitable syringe volume may range, for example, from 5 cc or less to 50 cc or more, typically from 5 cc to 15 cc. In addition to the composition comprising the iodinated polysaccharide, the medical kits may include one or more of the following: (a) an injectable degradative composition in a container (e.g., in dried form or in a form ready for injection), the degradative composition being one that breaks down the iodinated polysaccharide (e.g., hyaluronidase for a hyaluronic acid containing polysaccharides), (b) a catheter or other delivery device, (b) a needle, or (d) a diluent fluid suitable for injection (e.g., water for injection or saline).

What is claimed is:

1. An iodinated polysaccharide compound that comprises a polysaccharide backbone that comprises a plurality of carboxyl groups and a plurality of iodinated side groups.

2. The iodinated polysaccharide compound of claim 1, wherein the polysaccharide backbone comprises a carboxyl-containing polysaccharide chain to which the iodinated side groups are attached, the carboxyl-containing polysaccharide chain comprising one or more residues selected from one or more of glucuronic acid residues, mannuronic acid residues, or galacturonic acid residues.

3. An iodinated polysaccharide compound in which at least a portion of carboxyl groups that are present in a carboxyl-containing polysaccharide chain are functionalized with a plurality of iodinated side groups.

4. The iodinated polysaccharide compound of claim 3, wherein the carboxyl-containing polysaccharide chain comprises one or more residues selected from glucuronic acid residues, mannuronic acid residues, or galacturonic acid residues.

5. The iodinated polysaccharide compound of claim 1, wherein the iodinated side groups comprise an iodinated aromatic group in which one or more hydrogens of an aromatic group is substituted by iodine and one or more hydrogens of the aromatic group is substituted by a hydrophilic group.

6. The iodinated polysaccharide compound of claim 5, wherein the aromatic group is a phenyl group.

7. The iodinated polysaccharide compound of claim 1, wherein the iodinated side groups comprise an iodinated aromatic group and a hydrophilic group.

8. The iodinated polysaccharide compound of claim 7, wherein the iodinated aromatic side groups comprise a monoiodo-phenyl group, a diiodo-phenyl group, a triiodo-phenyl group or a tetraiodo-phenyl group.

9. The iodinated polysaccharide compound of claim 5, where the hydrophilic group comprises a polyhydroxylated group.

10. The iodinated polysaccharide compound of claim 9, wherein the polyhydroxylated group comprises a polyhydroxylated-C₁-C₆-alkyl-containing group.

11. The iodinated polysaccharide compound of claim 1, wherein the iodinated side groups comprise a 2,4,6-triiodobenzene group in which at least one of the hydrogens at the 3 and 5 positions is substituted by a polyhydroxylated group.

12. The iodinated polysaccharide compound of claim 1, wherein the iodinated side groups comprise an —N,N′-bis(polyhydroxy-C₁-C₆-alkyl)-2,4,6-triiodobenzene-3,5-dicarboxamide group.

13. A method of forming the iodinated polysaccharide compound in accordance with claim 1, comprising forming an amide linkage by a coupling reaction in which an amino group of an amino-containing iodinated compound is reacted with carboxyl groups of the carboxyl-containing polysaccharide chain.

14. The method of claim 13, wherein the coupling is performed in aqueous solution in the presence of a coupling agent.

15. The method of claim 14, wherein the coupling agent is a carbodiimide coupling agent.

16. The method of claim 13, wherein the amino-containing iodinated compound comprises an aromatic group in which one or more hydrogens is substituted by an amino-containing group, one or more hydrogens is substituted by iodine and one or more hydrogens is substituted by a hydrophilic group.

17. A medical composition comprising the iodinated polysaccharide compound in accordance with claim 1.

18. A medical procedure comprising introducing a medical composition of claim 17 into or between tissue of a patient.

19. The medical procedure of claim 18, further comprising imaging the medical composition using an x-ray-based imaging technique.

20. The medical procedure of claim 18, wherein the medical procedure is selected from a procedure to implant a fiducial marker comprising the iodinated polysaccharide compound, a procedure to implant a tissue regeneration scaffold comprising the iodinated polysaccharide compound, a procedure to implant a tissue support comprising the iodinated polysaccharide compound, a procedure to implant a tissue bulking agent comprising the iodinated polysaccharide compound, a procedure to implant a therapeutic-agent-containing depot comprising the iodinated polysaccharide compound, a tissue augmentation procedure comprising implanting the medical composition, a procedure to introduce the medical composition between a first tissue and a second tissue to space the first tissue from the second tissue.
